(19) United States
(12) Patent Application Publication
(10) Pub. No.: US 2010/0186753 A1
(43) Pub. Date: Jul. 29, 2010

(54) Sani-Thyro Cover for Thyroid Shields

(76) Inventor: Bernadette Buckley, Mattituck, NY (US)

Correspondence Address: Bernadette Buckley, 9180 Old Sound Ave, Mattituck, NY, 11952, US

(21) Appl. No.: 12/655,548

(22) Filed: Mar. 1, 2010

Related U.S. Application Data
(60) Provisional application No. 61/205,694, filed on Jan. 23, 2009.

(51) Int. Cl. A61B 19/02

(52) U.S. Cl. 128/849

(57) ABSTRACT

Thyroid shield covers will be used to improve overall sanitary conditions in the field of Radiology. The Sani-Thyro thyroid shield cover will be used to protect lead thyroid shields from becoming soiled with blood and other bodily fluids during Radiological procedures in which lead apparel is necessary. Lead apparel is expensive and therefore re-used throughout the lifetime of the integrity of the lead itself. The more often used, lead apparel becomes quite soiled over time. These thyroid shield covers will keep the thyroid shields clean and therefore help maintain personal hygene for all in the field of Radiology.

A provisional patent for this invention was filed on Jan. 23, 2009. The application number for this provisional patent is No. 61/205,694. A non-provisional patent is sought for sanitary thyroid shield covers. A follow up non-provisional application was filed within the year time limit permitted to do so. The application number for this non-provisional application is Ser. No. 12/655,548.

Radiological Technologists and Operating Room personnel use lead apparel for protection against X-rays needed during surgical procedures. Dentists also use lead apparel for the protection for their patients during dental X-ray examinations. Being a Radiological Technologist, I have experienced the unpleasant issue of having to use a thyroid shield on the job which had been previously used by an undetermined number of people. Lead protective gear is expensive and therefore used over frequently by personnel and patients, which causes it to become soiled and stained in the process.

The disposable Sani-Thyro thyroid shield covers will protect frequently used thyroid shields from becoming soiled with blood and other bodily fluids and will be discarded after each use as is the case with other surgical apparel. These disposable thyroid shield covers will be made of surgical apparel paper such as surgical gowns or masks already used in the field.

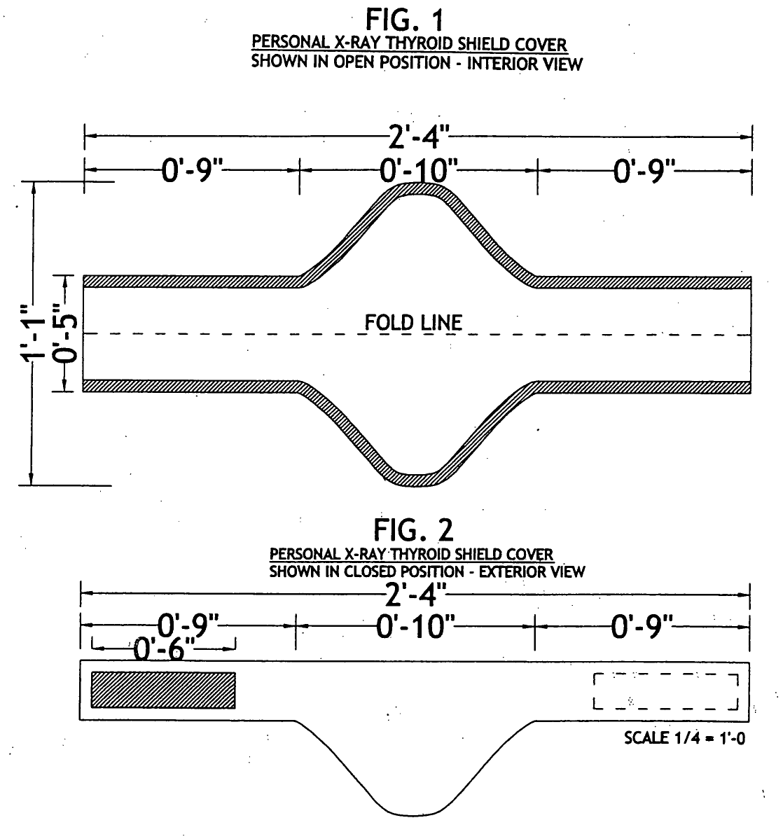

FIG. 1 on both pages 1/1 and 1/2, shows an open thyroid shield cover. The fold line is indicated by a dashed line across the middle of the thyroid shield cover.

FIG. 2, on both pages 1/1 and 1/2, shows the thyroid shield cover in a closed position.

The disposable thyroid shield cover in FIG. 1 on page 1/2, will have a self adhesive strip along its edges to adhere to itself when folded over and around the thyroid shield. This self adhesive is shown by grey hatched lines. The grey shaded lines in FIG. 2 on page 1/2 indicates a peel and stick type fastening system on the outside of the folded thyroid shield cover, which adheres to itself and is fastened around the back of the neck of an individual.

The thyroid shield covers on page 2/2 are personal thyroid shield covers and will be made of cotton cloth, washable and therefore reusable by the owner. The grey shaded areas in FIGS. 1 and 2 on page 2/2 indicate a hook and loop type fastening system.

The fact of not knowing how clean or soiled a thyroid shield is following multiple uses is what prompted my idea of thyroid shield covers back in the year 2003. I feel that these sanitary thyroid shield covers (be they disposable or personal) for Radiology personnel and patients as well, will greatly improve overall sanitary conditions and public health.

1. I claim the invention of thyroid shield covers which will be used in the field of Radiology to protect lead thyroid shields from being soiled. My invention originated in the year 2003 while working in the field of Radiology. The name of my invention will be the Sani-Thyro thyroid shield cover. I believe I am the original and first inventor of the thyroid shield cover and claim it as my own. The sanitary thyroid shield cover will be used to improve overall sanitary conditions during exams and surgeries in the Radiological field for both Radiology personnel and their patients. A non-provisional patent is sought for this patent application Ser. No. 12/655,548. Disposable thyroid shield covers will be made of a surgical paper, such as surgical apparel already used in the field of Radiology. Personal thyroid shield covers will be made of cotton material.
